(19) United States
(12) Patent Application Publication
(10) Pub. No.: US 2024/0299038 A1
(43) Pub. Date: Sep. 12, 2024

(54) AUTOMATIC TOURNIQUET AND AUTOMATIC PREVENTION OF BLEEDING USING SAME

(71) Applicant: XMETIX LTD., Nazareth (IL)

(72) Inventors: Irad KUHNREICH, Haifa (IL); Erez YEHEZKELY, Zichron Yaakov (IL); German SHIKLOV, Haifa (IL)

(73) Assignee: XMETIX LTD., Nazareth (IL)

(21) Appl. No.: 18/281,335

(22) PCT Filed: Mar. 11, 2022
(86) PCT No.: PCT/IL2022/050280

§ 371 Date: Sep. 11, 2023

Related U.S. Application Data
(60) Provisional application No. 63/159,629, filed on Mar. 11, 2021.

(51) Int. Cl. A61B 17/132; A61B 17/00; A61B 90/00; B64G 6/00

(52) U.S. Cl. CPC A61B 17/1327; A61B 90/06; A61B 2017/00017; A61B 2090/064; B64G 6/00

(57) ABSTRACT

An automatic pressure drop prevention device is disclosed. The automatic pressure drop prevention device comprises: at least one strip wherein the at least one strip is selected from; a wire, a band, and a combination thereof; and an actuator. The actuator may include: an electric motor; and a gear assembly, such that, the strip is anchored to the gear assembly in at least two places, to create a loop, and wherein the gear assembly is configured to tighten the loop when actuated in a first direction, and to untighten the loop when actuated in a second direction.

### CROSS-REFERENCE TO RELATED APPLICATIONS

This application is an International Patent application which claims the benefit of priority of U.S. Provisional Patent Application No. 63/159,629, entitled “AUTOMATIC TOURNIQUET AND AUTOMATIC PREVENTION OF BLEEDING USING SAME”, filed Mar. 11, 2021, which is hereby fully incorporated by reference in its entirety.

### FIELD OF THE INVENTION

The present invention relates generally to the prevention of bleeding using a tourniquet. More specifically, the present invention relates to an automatic prevention of bleeding using an automatic tourniquet.

### BACKGROUND OF THE INVENTION

Tourniquets are medical devices that are used to apply pressure to a limb of a patient in order to limit, but not stop, the flow of blood. Tourniquets are useful in extreme medical conditions, such as, limb injuries and operations, but, also for determining the location of a suitable vein for venipuncture. All known tourniquets are manually operated and adjusted, usually by a professional, such as, a medic, a paramedic, a nurse, or a physician.

In some emergency occasions, for example, on a battlefield or a car accident, a critical time may pass, until professional assistance is provided to an injured patient. When a nonprofessional may try to apply a tourniquet, he/she may cause irreversible damage or not apply sufficient pressure to stop bleeding. For example, experiments conducted with a combat application tourniquet (CAT) yielded that the time for placement of the tourniquet on the limb varies between 43 sec. for a non-trained user, 38 sec. for a trained user, and 58 for a trained user under pressure. Furthermore, the blood pulse elimination was at most 67% although the CAT is capable of better pulse elimination.

Accordingly, there is a need for an automatic tourniquet that may be safely activated by a nonprofessional. Such a tourniquet can be automatically operated, following a signal related to the blood flow, thus may save critical time during injury.

### SUMMARY OF THE INVENTION

Some aspects of the invention may be directed to an automatic pressure drop prevention device comprising: at least one strip wherein the at least one strip is selected from; a wire, a band, and a combination thereof, and an actuator. In some embodiments, the actuator may include: an electric motor; and a gear assembly, wherein the strip is anchored to the gear assembly in at least two places, to create a loop, and wherein the gear assembly is configured to tighten the loop when actuated in a first direction, and to untighten the loop when actuated in a second direction.

In some embodiments, the electric motor is a DC electric motor. In some embodiments, the gear assembly comprises a planetary element connected to a worm gear. In some embodiments, the strip is threaded in at least two groves in the planetary element. In some embodiments, the strip is connected to the planetary element in at least two locations.

In some embodiments, the electrical motor is controlled to provide rotary torque to the gear assembly to cause the tourniquet band to form a target pressure on a limb. In some embodiments, the gear assembly is designed to cause the tourniquet band to form a target pressure on the limb.

In some embodiments, the automatic pressure drop prevention device may further include one or more sensors selected from: a pressure sensor, an attachment sensor, a force sensor, a thermometer, an accelerometer, and the like) and the electrical motor may be controlled to provide rotary torque to the gear assembly based on a signal received from the one or more sensors.

In some embodiments, the electrical motor is controlled to provide rotary torque to the gear assembly, in a predetermined sequence.

In some embodiments, the automatic pressure drop prevention device may further include a manual fast release unit. In some embodiments, the automatic pressure drop prevention device may further include a manual pressure-built unit.

Some aspects of the invention may be directed to a garment comprising the automatic pressure drop prevention device according to embodiments of the invention.

Some additional aspects of the invention may be directed to a method for automatic prevention of pressure drop using an automatic pressure drop prevention device, the method may include: providing an automatic pressure drop prevention device to be worn on a user's limb; and upon detecting a pressure drop, automatically activating the automatic pressure drop prevention device to stop the bleeding. In some embodiments, the automatic pressure drop prevention device may include at least one strip wherein the at least one strip is selected from; a wire, a band, and a combination thereof; and an actuator. In some embodiments, the actuator may include: an electric motor; and a gear assembly, wherein the strip is anchored to the gear assembly in at least two places, to create a loop, and wherein the gear assembly is configured to tighten the loop when actuated in a first direction, and to untighten the loop when actuated in a second direction.

In some embodiments, detecting the pressure drop may include detecting an injury in the limb is from a signal received from one or more sensors. In some embodiments, detecting the pressure drop may include detecting a pressure drop in a spacesuit.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is an illustration of an automatic pressure drop prevention device applied on a limb according to some embodiments of the invention;

FIGS. 1B and 1C are illustrations of a front view and top view of an actuator according to some embodiments of the invention;

FIG. 2 is an illustration of another automatic pressure drop prevention device according to some embodiments of the invention; and

FIG. 3 is a flowchart of a method for automatic prevention of pressure drop using the automatic pressure drop prevention device according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of the same or similar features or elements may not be repeated.

Some aspects of the invention may be directed to an automatic pressure drop prevention device, for example, an automatic tourniquet, and a method for automatic prevention of pressure drop, for example, bleeding using the automatic tourniquet. Such an automatic tourniquet may be applied and activated at need (e.g., during injury) by a nonprofessional. In another example, the automatic pressure drop prevention device may be applied on a spacesuit and activated at need, for example, during a pressure drop due to leakage in the suit during a spacewalk. Additionally or alternatively, the automatic be applied and activated at need may be worn by a potential user (e.g., a soldier on a battlefield, an astronaut) in a released state, for example, above or below the uniform. Therefore, in a case of an injury, the automatic tourniquet may be activated by the user/a fellow soldier or may be self-activated following, for example, an indication of blood pressure drops in the vicinity of the automatic tourniquet or indication of a pressure drop in a spacesuit.

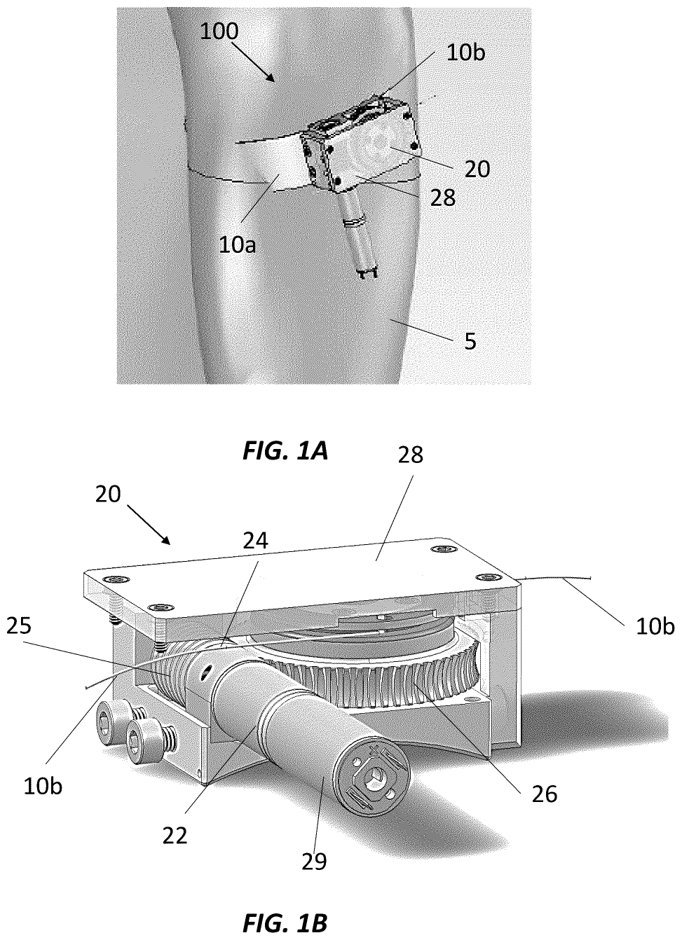

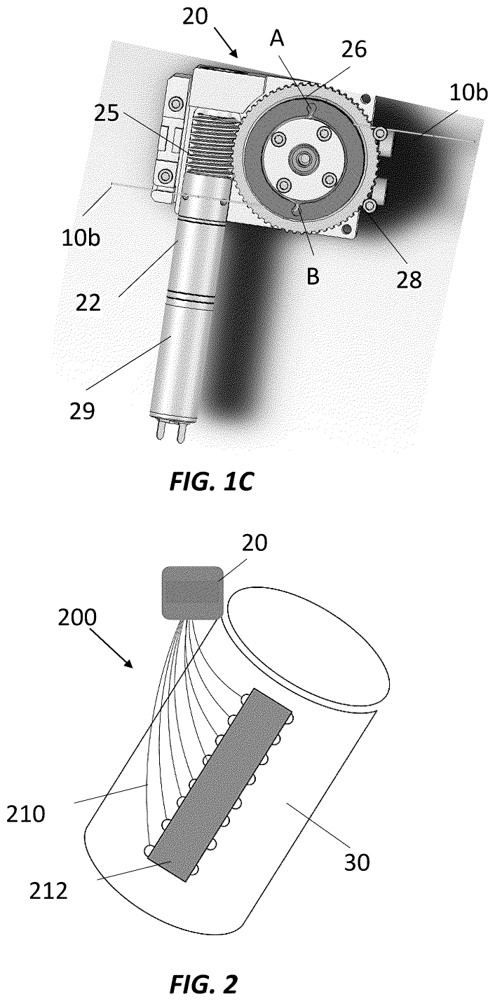

Reference is now made to FIG. 1A which is an illustration of an automatic pressure drop prevention device (e.g., a tourniquet) applied on a limb according to some embodiments of the invention. An automatic pressure drop prevention device 100 may be applied on a limb 5 (e.g., leg, arm, etc.) of a patient/user. Automatic pressure drop prevention device 100 may include at least one strip, the at least one strip is selected from; a wire 10b, a band 10a, and a combination thereof. For example, wire 10b may be connected at two ends to band 10a, as illustrated. In some embodiments, more than one strip (e.g., wire) may be included in automatic pressure drop prevention device 100 as illustrated and discussed with respect to FIG. 2. Wire 10b may be any suitable ridged wire, for example, a metallic wire, and band 10b may be any suitable band, for example, a band used in commercial tourniquets (e.g., CAT) having a width of approximately 25 mm.

In some embodiments, wires 10a and/or band 10b may be threaded/attached/at least partially glued etc. to the uniform/garment worn by a potential user. In some embodiments, a housing 28 may be attached to the uniform/garment. These options may save critical time during injury, by allowing automatic pressure drop prevention device (e.g., tourniquet) 100 to be instantly activated during the injury, without the need for placement on limb 5.

In some embodiments, automatic pressure drop prevention device 100 may further include an actuator 20 at least partially held in housing 28. Actuator 20 may be configured to tighten a loop formed by the strip when actuated in a first direction and to untighten the loop when actuated in a second direction. The loop may warp limb 5.

Reference is now made to FIGS. 1B and 1C which are illustrations of a front view and top view of an actuator according to some embodiments of the invention. Actuator 20 may include an electric motor 22 (e.g., a DC electric motor) and a gear assembly 24. In some embodiments, strip 10a and/or 10b may be anchored to gear assembly 24 in at least two places ‘A’ and ‘B’, to create the loop, and gear assembly 24 may be configured to tighten the loop when actuated in a first direction, and to untighten the loop when actuated in a second direction. In a nonlimiting example, gear assembly 24 may cause a tightening pressure of 300 mm Hg on a limb within less than 30 sec by causing band 10b and/or wire 10a to travel approximately 80 mm.

In some embodiments, gear assembly 24 may include a planetary element 26 (e.g., the illustrated planetary gear) connected to a worm gear 25. In some embodiments, the strip (e.g., wire 10b) may be threaded in at least two groves in planetary element 26, for example, in locations ‘A’ and ‘B’. In some embodiments, the strip may be connected in at least two locations in planetary element 26, for example, locations ‘A’ and ‘B’. In some embodiments, other gear assemblies may be used for transferring the motion from motor 22 to the strip, for example, a miter gear, spiral bevel gear, straight bevel gear, rack and pinion, lead screw, etc.

In some embodiments, motor 22 and gear assembly 24 may be at least partially held by housing 28.

In some embodiments, some or all of the components of actuator 20 may be made from a rigid polymer, for example, gear assembly 24 and housing 28. A ridged polymer may be defined as a polymer having a tensile strength of at least Strength 11,000 psi, for example, acetal homopolymer.

In some embodiments, electrical motor 22 may be controlled to provide rotary torque to gear assembly 24 to cause tourniquet 100 to form a target pressure on limb 5, for example, between 270-300 mm Hg. In some embodiments, gear assembly 24 may be designed to cause tourniquet 100 to form the target pressure on limb 5, in less than 30 sec. At the pressure pick, the tension on the strip may be approximately 10 N.

In some embodiments, automatic tourniquet 100 may further include one or more sensors (not illustrated) and electrical motor 22 may be controlled to provide rotary torque to gear assembly 26 based on a signal received from the one or more sensors. The one or more sensors may be selected from: a pressure sensor, an attachment sensor, a thermometer, an accelerometer, a force sensor, and the like. A nonlimiting example for a pressure sensor may include a flexible sensor, such as, a piezoelectric sensor, a flexible force-sensitive resistor and the like. Such a flexible sensor may easily fit or adjust to the shape of the limb, thus may be assembled on the strip. Additionally or alternatively the one or more sensors may be attached to a face of housing 28 facing limb 5 when automatic pressure drop prevention device 100 is applied on limb 5. In some embodiments, automatic pressure drop prevention device 100 may further include a controller (e.g., a chip assembled on a PCB) that is configured to received signals from the one or more sensors. In a nonlimiting example, a pressure sensor or a force sensor may allow monitoring the blood pressure in limb 5 and may activate motor 22 when a severe blood pressure drop (e.g., above a threshold value) is detected. In another nonlimiting example, the sensor (e.g., a pressure sensor or a force sensor) may allow monitoring the post pressure application, alerting if any drop in the applied pressure is detected, for example, due to movement of the limb. In a nonlimiting example, the movement of the limb may be detected by an accelerometer. In such a case, the controller may control motor 22 to provide the additional torque required to reestablished the target pressure.

In some embodiments, electrical motor 22 may be controlled to provide rotary torque to gear assembly 24 in a predetermined sequence. For example, motor 22 may be configured to apply the pressure in small steps (e.g., each step corresponds to one rotation of the motor's axis) while applying 5 steps forward in the tightening direction and 2 steps backwards in the untightening direction until reaching the target pressure. This form of operation may allow trapped body organs to be released.

In some embodiments, automatic pressure drop prevention device 100 may further include a battery 29 (e.g., a lithium battery) for providing electrical energy to motor 22 and optionally also to the one or more sensors and the controller.

In some embodiments, automatic pressure drop prevention device 100 may further include a manual fast release unit (not illustrated). In case of a false positive situation when the tourniquet is activated when it is not needed, the user may have the option to immediately release the tourniquet pressure. For example, the user may cut band 10a/wires 10b. In another example, the user may pull a strap connected to a cutting knife pre-installed in the system or may pull a pin disassembling the gear.

In some embodiments, automatic pressure drop prevention device 100 may further include a manual pressure buildup, in case motor 22 is not working. Automatic pressure drop prevention device 100 may have an option for manual pressure buildup based on pulling a strap or rotating a handle.

Reference is now made to FIG. 2 which is an illustration of another automatic pressure drop prevention device according to some embodiments of the invention. An automatic pressure drop prevention device 200 may include substantially the same actuator 20 as automatic pressure drop prevention device 100. In some embodiments, a plurality of wires 210 may be anchored to actuator 20 forming an array of wires 210 wrapping a sleeve 30 or a garment. In some embodiments, wires 210 may be threaded on a strip 212 attached to sleeve 30. Such a tourniquet may provide the required pressure to a wider portion of the limb.

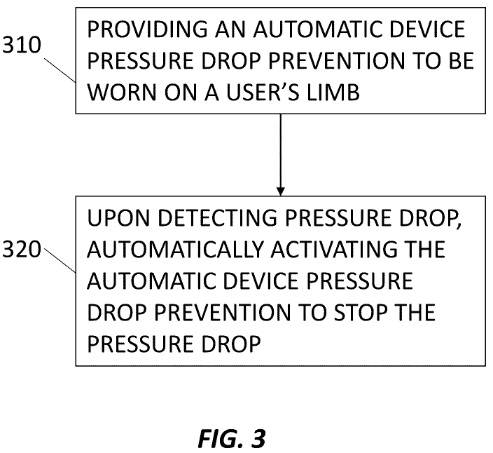

Reference is now made to FIG. 3 which is a flowchart of a method for automatic prevention of pressure drop using an automatic pressure drop prevention device according to some embodiments of the invention. In step 310, an automatic tourniquet may be provided to be worn on a user's limb. For example, automatic pressure drop prevention device 100 or 200 may be pre-placed/attached to a garment worn by the user (e.g., uniform, spacesuit, etc.). In step 320, upon detecting a pressure drop (e.g., an injury in the limb), the automatic pressure drop prevention device may be automatically activated to stop the pressure drop (e.g., bleeding). For example, actuator 20 may be activated to cause the strip(s) to be tightened to a required pressure around limb 5. In some embodiments, actuator 20 may be automatically activated following a signal received from one or more sensors, for example, indicating a drop in the blood pressure, movement of the limb, and the like.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. An automatic pressure drop prevention device, comprising:
at least one strip wherein the at least one strip is selected from; a wire, a band and a combination thereof, and
an actuator,
wherein the actuator comprises:
an electric motor; and
a gear assembly, wherein the strip is anchored to the gear assembly in at least two places, to create a loop, and wherein the gear assembly is configured to tighten the loop when actuated in a first direction, and to untighten the loop when actuated in a second direction.

2. The automatic pressure drop prevention device of claim 1, wherein the pressure drop is a blood pressure drop and the device is an automatic tourniquet.

3. The automatic pressure drop prevention device of claim 1, wherein the pressure drop is an air pressure leak from a spacesuit and the device is a safety device.

4. The automatic pressure drop prevention device, according to claim 1, wherein the electric motor is a DC electric motor.

5. The automatic pressure drop prevention device, according to claim 1, wherein the gear assembly comprises a planetary element connected to a worm gear.

6. The automatic pressure drop prevention device of claim 5, wherein the strip is threaded in at least two groves in the planetary element.

7. The automatic pressure drop prevention device of claim 5, wherein the strip is connected to the planetary element in at least two locations.

8. The automatic pressure drop prevention device according to claim 1, wherein the electrical motor is controlled to provide rotary torque to the gear assembly to cause the tourniquet band to form a target pressure on a limb.

9. The automatic pressure drop prevention device according to claim 1, wherein the gear assembly is designed to cause the tourniquet band to form a target pressure on a limb.

10. The automatic pressure drop prevention device according to claim 1, further comprising one or more sensors, and wherein the electrical motor is controlled to provide rotary torque to the gear assembly based on a signal received from the one or more sensors.

11. The automatic pressure drop prevention device according to claim 1, wherein the electrical motor is controlled to provide rotary torque to the gear assembly, in a predetermined sequence.

12. The automatic pressure drop prevention device according to claim 1, further comprising a manual fast release unit.

13. The automatic pressure drop prevention device according to claim 1, further comprising a manual pressure-built unit.

14. The automatic pressure drop prevention device according to claim 1, wherein the device is integrated in a garment.

15. A method for automatic prevention of pressure drop using an automatic pressure drop prevention device, the method comprising:
providing an automatic pressure drop prevention device to be worn on a user's limb; and
upon detecting a pressure drop, automatically activating the automatic pressure drop prevention device to stop the pressure drop,
wherein the automatic pressure drop prevention device comprises:
a strip wherein the strip is selected from; a wire, a band and a combination thereof, and
an actuator,
wherein the actuator comprises:
an electric motor; and
a gear assembly, wherein the strip is anchored to the gear assembly in at least two places, to create a loop, and wherein the gear assembly is configured to tighten the loop when actuated in a first direction, and to untighten the loop when actuated in a second direction.

16. The method of claim 15, wherein detecting the pressure drop comprises detecting an injury in the limb associated with a blood pressure drop, is from a signal received from one or more sensors.

17. The method of claim 15, wherein detecting the pressure drop comprises detecting a pressure drop in a spacesuit.
